(19) United States
(12) Patent Application Publication
(10) Pub. No.: US 2018/0311395 A1
(43) Pub. Date: Nov. 1, 2018

(54) PERFUME COMPOSITIONS AND AIR FRESHENER DEVICE

(71) Applicant: Givaudan, S.A., Vernier (CH)

(72) Inventors: Philippe BLONDEAU, Paris (FR); Sebastien PICCI, Kingersheim (FR)

(21) Appl. No.: 15/770,574

(22) PCT Filed: Nov. 16, 2016
(86) PCT No.: PCT/EP2016/077825

§ 371 Date: Apr. 24, 2018

(30) Foreign Application Priority Data
Nov. 17, 2015 (GB) 1520246.8

(51) Int. Cl. A61L 9/12; A61L 9/01

(52) U.S. Cl. CPC A61L 9/12; A61L 2209/131; A61L 9/01

(57) ABSTRACT

A perfume composition for use in non-porous membrane-based air fresheners is provided. The composition includes one or more fragrance ingredients; and from about 5% to about 90% by weight of a liquid carrier having individual Hansen solubility parameters of: a dispersion solubility parameter (δ_{d}) less than about 17 MPa^{1/2}, a polar solubility parameter (δₚ) ranging from about 1 to about 7 MPa^{1/2}, and a hydrogen bond solubility parameter (δₕ) ranging from about 2 to about 7 MPa^{1/2}; a C log P lower than about 3.5; and a vapor pressure ranging from about 0.2 to about 1 mmHg.

### FIELD OF THE INVENTION

The present disclosure relates to perfume compositions. More particularly, the present disclosure relates to perfume compositions for use in non-porous polyolefin membrane-based air fresheners, the perfume compositions having certain liquid carriers that improve the release of fragrance ingredients into the air.

### BACKGROUND OF THE INVENTION

Membrane-based air freshener devices that deliver a pleasant fragrance into the air are well known. These devices consist of a housing having a fragrance reservoir or chamber including a perfume composition, sealed with a membrane which is permeable to perfume vapors. The membrane is usually made of non-porous polyolefin (also called dense polyolefin), such as polyethylene or polyethylene composites, but other materials are also possible. The device can be used at room temperature (passive mode) or heated to promote fragrance evaporation, or submitted to an air flow to entrain the perfume vapor through the membrane (active mode). However, more generally, membrane-based air freshener devices are used under passive mode. As such, the primary driving force that makes the fragrance ingredients diffuse through the membrane is the volatility of the fragrance ingredients themselves. While this works, passive membrane devices suffer from the problem that typically such devices must include at least 70 wt % of fragrance ingredients having a vapor pressure of at least 0.004 mmHg at 25° C. A principal limitation of membrane-based air freshener devices is the fact that among all fragrance ingredients having the same volatility, some fragrance ingredients diffuse easily and rapidly through the membrane, while other fragrance ingredients do not diffuse of diffuse very slowly. This leads to undesired distortion of the perfume character. For example, the perfume note may change, or become unbalanced and hedonically unacceptable. Furthermore, fragrance ingredients that do not diffuse or diffuse slowly through the membrane accumulate in the fragrance reservoir and form residues that remain in the reservoir after the air freshener device has stopped working, for example after 30 days.

Adding iso-paraffinic oils, alone or in combination with polar solvents, referred to as “liquid carriers” hereafter, to the perfume oil, in order to enhance both the evaporation of fragrance ingredients and the transport of the latter through non-porous polyolefin membranes is an established method. However, such oils may have the detrimental effect of inducing membrane delamination from the reservoir blister, leading to potential liquid perfume leakage during use. This is highly undesirable and limits the level of such iso-paraffinic oils that can be practically used in these devices to not more than 35 wt %. Furthermore, the use of hydrocarbons is also limited to 10 wt % in some countries, due to regulatory constraints.

Further, adding high levels of fragrance ingredients, selected from aromatic esters, such as BENZYL ACETATE, or alkyl esters derived from alkyl alcohols and/or alcohols derived from terpenes, such as ISONONANYL ACETATE, LINALYL ACETATE and TERPENYL ACETATE as alternative liquid carriers in order to enhance both the evaporation of fragrance ingredients and the transport of the latter through non-porous polyolefin membranes is another established method. However, such liquid carriers have significant odor and therefore cannot be used in all perfume compositions. Furthermore, the use of esters does not resolve the problem of membrane selectivity with respect to certain problematic fragrance ingredients, such as alcohols.

Accordingly, it would be desirable to provide a liquid carrier that enhances the evaporation and permeation of the broadest possible selection of fragrance ingredients in the required volatility range for passive membrane-based air freshener applications, while having no impact on membrane/blister seal.

### SUMMARY OF THE INVENTION

In one embodiment, a perfume composition for use in non-porous polyolefin membrane-based air fresheners includes one or more fragrance ingredients; and from about 5% to about 90% by weight of a liquid carrier having individual Hansen solubility parameters of: a dispersion solubility parameter (δ_{d}) less than about 17 MPa^{1/2}, a polar solubility parameter (δₚ) ranging from about 1 to about 7 MPa^{1/2}, and a hydrogen bond solubility parameter (δₕ) ranging from about 2 to about 7 MPa^{1/2}; a C log P lower than about 3.5; and a vapor pressure ranging from about 0.2 to about 1 mmHg.

In another embodiment, an air freshener device includes a housing; and a reservoir including a perfume composition and a permeable membrane through which a vapor of the perfume composition can permeate. The perfume composition includes one or more fragrance ingredients; and from about 5% to about 90% by weight of a liquid carrier having individual Hansen solubility parameters of: a dispersion solubility parameter (δ_{d}) less than about 17 MPa^{1/2}, a polar solubility parameter (δₚ) ranging from about 1 to about 7 MPa^{1/2}, and a hydrogen bond solubility parameter (δₕ) ranging from about 2 to about 7 MPa^{1/2}; a C log P lower than about 3.5; and a vapor pressure ranging from about 0.2 to about 1 mmHg.

In yet another embodiment, a perfume composition includes at least about 25% by weight of one or more fragrance ingredients; and from about 10% to about 60% by weight of a liquid carrier. The one or more fragrance ingredients is selected from the group consisting of alkyl alcohols having a chain length larger than about 5 carbon atoms, terpene alcohols, and alcohols derived from modified terpenes or terpene analogues. The liquid carrier is dipropylene glycol dimethyl ether (DPGDME).

These and other features, aspects and advantages of specific embodiments will become evident to those skilled in the art from a reading of the present disclosure.

### DETAILED DESCRIPTION OF THE INVENTION

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

The present disclosure relates to perfume compositions including a mixture of one or more fragrance ingredients and a liquid carrier; as well as membrane-based air fresheners containing the same.

In one embodiment, the present disclosure relates to air fresheners having a membrane for which fragrance ingredients having similar vapor pressure but different chemical properties have different permeation rates through the membrane. A non-limiting example of such a membrane is one made of non-porous polyethylene. Non-limiting examples of fragrance ingredients having similar vapor pressure but different chemical properties are DIHYDROMYRCENOL (2,6-dimethyloct-7-en-2-ol) and ALLYL OENANTHATE (allyl heptanoate). For the sake of the present disclosure, the permeation rate of a perfumery ingredient through the membrane or a membrane air freshener device is taken as the initial slope of the weight loss of the air freshener device as a function of time and is expressed in weight percentage (wt %)/day unit. For example, the permeation rate of DIHYDROMYRCENOL through a membrane consisting entirely of polyethylene is typically less than 0.5 wt %/day, whereas the permeation rate of ALLYL OENANTHATE is typically higher than 2 wt %/day through the same membrane. This leads to a maximal DIHYDROMYRCENOL to ALLYL OENANTHATE permeation ratio of 0.25, which is far below 1. This means that the composition of a mixture of DIHYDROMYCENOL and ALLYL OENANTHATE permeating through a membrane would significantly change with time, so that the level of ALLYL OENANTHATE would be rapidly depleted while that of DIHYDROMYRCENOL would increase. This would cause the odor character of the composition to change with time in an unacceptable manner to consumers.

Whatever the particular design of the membrane-based air freshener devices according to the present disclosure, it is important that the devices perform in a manner that allows fragrance material to be dispensed at a steady and controlled rate into an environment.

In one embodiment, the efficiency of a membrane-based air freshener device is determined by the amount of perfume released to the atmosphere during a certain period of time, for example, between about 30 and about 50 days. During this period of time, which is also referred to as the lifetime of the air freshener device, it is expected that the following conditions are fulfilled: (i) the rate of perfume release into the atmosphere is continuous and nearly constant, i.e. the rate release does not change significantly with time, (ii) the profile of the perfume remains nearly constant, i.e. the odor character of the perfume remains essentially unchanged over time and (iii) the perfume is released in such a way that the totality of the perfume has evaporated during the lifetime of the air freshener, i.e. there is no perfume residue left in the device. In other words, the ratio of the permeation rate of any fragrance ingredient in the perfume to any of the other fragrance ingredients of the perfume should be as close to 1 as possible.

However, experience shows that such conditions are not easily fulfilled for the reasons that fragrance ingredients differ in volatility and in chemical nature. Differences in volatility may lead to the exhaustion of the most volatile ingredients before the air freshener device has reached the end of its lifetime, which in turn may lead to changes in the odor character of the perfume. Conversely, fragrance ingredients having too low a volatility may not evaporate completely during the life of the air freshener device leaving residual material in the fragrance vessel. Differences in chemical nature may lead to different permeation rate though the membrane. In particular, the membrane may be impervious or only sparingly permeable to ingredients that are not soluble in or compatible with the membrane. This may also lead to changes in the odor character of the emitted perfume and to the occurrence of residues in the fragrance vessel after the lifetime of the air freshener device has ended. For example, the most problematic fragrance ingredients in terms of permeation behavior through non-porous polyolefin-based membranes, such as non-porous polyethylene and non-porous polypropylene-based membranes are linear and branched alcohols, such as n-alkyl alcohols, iso-alkyl alcohols and alcohols derived from terpenes, for example NONANOL, DIHYDROMYRCENOL, LINALOOL, and the like.

In addressing the shortcomings in the art, applicants surprisingly found that saturated hydrocarbons have detrimental impact on the seal joining the polyethylene membrane to the blister comprising the fragrance reservoir, resulting in leakage of fragrance through the seal and even breakage or delamination of the seal. Additionally, applicants have found that using polar solvents alone does not solve the hereinabove mentioned selectivity issues, mainly because polar solvents do not evaporate as well as apolar solvents and because polar solvents are not compatible with the polyethylene membrane and are therefore inefficient in controlling the permeation of fragrance ingredients.

### Perfume Compositions

According to the present disclosure, perfume compositions include a liquid carrier and a mixture of one or more fragrance ingredients. These perfume compositions may be used in membrane-based air freshener devices and other similar consumer products.

Liquid Carrier

According to the present disclosure, the issues described above may be substantially resolved by using liquid carriers having particular combinations of Hansen solubility parameters and vapor pressure. The Hansen solubility parameters reflect how a particular chemical molecule interacts with neighboring molecules, also referred to as “environment”. In other words, the Hansen solubility parameters are a way of predicting if one material will dissolve in another and form a solution. According to Hansen, the interaction of a molecule with its environment is the result of three contributing solubility parameters, i.e. each molecule is given three Hansen parameters: the dispersion solubility parameter δ_{d}, reflecting the extent of dispersive interactions between molecules, the polar solubility parameter δₚ, reflecting the extent of dipole-dipole interactions between molecules, and the H-bond solubility parameter δₕ, reflecting the extent of hydrogen bonding between molecules. The solubility parameters are expressed in MPa^{1/2} units, where MPa means mega-pascal. For a detailed discussion of the theory underlying the Hansen solubility parameters, see for example A. F. N. M Barton, “Handbook of solubility parameters and other cohesion parameters”, 2^{nd} edition, CRC Press, 1991.

Applicant has found that liquid carriers having the following solubility parameters are useful for solubilizing fragrance ingredients according to the present disclosure: a dispersion Hansen parameter δ_{d} less than about 17 MPa^{1/2}; polar Hansen parameter δₚ in the range of from about 1 to about 7 MPa^{1/2}, in another embodiment from about 1.5 to about 5 MPa^{1/2} and in yet another embodiment from about 1.8 to about 4 MPa^{1/2}; and H-bond Hansen parameter δₕ in the range of from about 2 to about 7 MPa^{1/2}; in another embodiment from about 2.5 to about 6 MPa^{1/2} and in yet another embodiment from about 3 to about 5 MPa^{1/2}.

Hydrophobicity of the liquid carrier can be measured using log P value, a physico-chemical property. The octanol/water partition coefficient (P) of a carrier solvent is the ratio between its equilibrium concentrations in octanol and in water. The log P values can also be very conveniently calculated using the fragment approach of Hansch and Leo and given as C log P. See A. Leo, Comprehensive Medicinal Chemistry, Vol 4, C. Hansch et al. p 295, Pergamon press, 1990 and given as c log P. According to the present disclosure, liquid carriers have a calculated octanol/water partition coefficient C log P less than 3.5, in another embodiment less than 2.5, and in yet another embodiment less than 1.0. In another embodiment, liquid carriers have a vapor pressure in the range of from about 0.2 to about 1 mmHg, in another embodiment from about 0.3 to about 0.8 mmHg and in yet another embodiment from about 0.4 to about 0.6 mmHg.

In the context of the present disclosure, the Hansen parameters were calculated using HSPiP software, version 4.0.07 (www.Hansen-Solubility.com); the C log P values were calculated using the software embedded in ChemDraw Ultra software, version 12.0.2.1076 (Cambridesoft Corp.); and the vapor pressures are taken from www.thegoodscentscompany.com.

Non-limiting examples of liquid carriers having suitable Hansen solubility parameters include fully alkylated alkylene glycols, according to formula (I), wherein R₁ is CH3 or H; R₂ is H if R₁ is CH₃ or R₂ is CH₃ if R₁ is H; R₃ is CH₃ or H; and R₄ is H if R₃ is CH₃ or R₄ is CH₃ if R₃ is H.

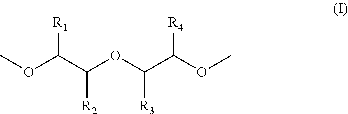

In one embodiment, the use of aprotic solvents, for example, fully alkylated alkylene glycols alone, i.e. without admixing saturated hydrocarbons, or any other apolar solvent to them, as a liquid carrier, facilitates the transfer of volatile ingredients through the membrane of an air freshener. Further, using aprotic solvents such as fully alkylated alkylene glycols as a liquid carrier facilitates the transfer of protic fragrance ingredients such as alcohols through the membrane, even if polar protic glycol ethers are not present. This is a considerable advantage of the present disclosure, considering the rising regulatory concerns impacting protic glycol ethers.

In one embodiment, perfume compositions according to the present disclosure include a liquid carrier comprising at least one aprotic fully alkylated alkylene glycols. In a particular embodiment, the level of fully alkylated alkylene glycols in the perfume composition may be from about 5 to about 90 wt %, in another embodiment from about 8 to about 70 wt %, and in yet another embodiment from about 10 to about 60 wt %.

In a further embodiment, the perfume composition may be substantially free of protic glycol ethers.

In one embodiment, the aprotic fully alkylated alkylene glycol ether liquid carrier is dipropylene glycol dimethyl ether (DPGDME), available under the trademark PROGLYDE DMM from Dow Chemicals.

Alternatively, Applicant has surprisingly discovered that fragrance ingredients having a dispersion Hansen parameter smaller than about 17 MPa^{1/2}, a polar Hansen parameter in the range of from about 1 to about 7 MPa^{1/2}, and a H-bond Hansen parameter in the range of from about 2 to about 7 MPa^{1/2}, together with a calculated octanol/water partition coefficient C log P lower than about 3.5 and a vapor pressure in the range of from about 0.2 to about 1 mmHg may also be used alone or in combination with DPGDME in membrane-based air freshener devices according to the present disclosure. Suitable fragrance ingredients include, but are not limited to, METHYL PAMPLEMOUSSE (6,6-dimethoxy-2,5,5-trimethylhex-2-ene); FRUCTONE (ethyl 2-(2-methyl-1,3-dioxolan-2-yl)acetate); ELINTAAL (3-(1-ethoxyethoxy)-3,7-dimethylocta-1,6-diene); LIFFAROME ((Z)-hex-3-en-1-yl methyl carbonate); and the like.

Optionally, the perfume composition may contain up to about 35 wt %, alternatively up to about 25 wt %, alternatively up to about 10 wt % of a second liquid carrier selected from iso-paraffins, also known as lightly hydrogenated petroleum distillates, which are complex mixtures of linear, branched and cyclic hydrocarbons with extremely low level of aromatic contaminants.

Suitable isoparaffinic hydrocarbon oils according to the present disclosure include, but are not limited to, C8-C9 Isoparaffin (Isopar® E, available from Exxon, CAS No. 70024-92-9); C8-C12 Isoparaffin (SHELL SOL TS, available from Shell); C10-11 Isoparaffin (Isopar® G, available from Exxon, CAS No. 246538-75-0); C10-12 Isoparaffin (SHELL SOL TD, available from Shell, CAS No. 68551-17-7); C11-C13 Isoparaffin (Isopar® L, available from Exxon, CAS No. 246538-78-3); C12-C14 Isoparaffin (SOLTROL 170 Isoparaffin Solvent, available from CPC chem, CAS No. 68551-19-9); C11-C16 Isoparaffin (Isopar® M, available from Exxon, CAS Number: 64742-47-8); Isoparaffin C12-C20 (Isopar® V Solvent, available from Exxon); and the like. For a more comprehensive list of isoparaffinic hydrocarbon oils and their properties, see “Wilbur Johnson, Jr. Green Book 5 of the Cosmetic Ingredient Review (CIR) Expert Panel, Aug. 30-31, 2010”, which is incorporated herein.

Isoparaffinic oils generally comprise high levels of branched hydrocarbons. For example, Isopar C consists of approximately 85 wt % isooctane, Isopar® L contains about 85 wt % of branched hydrocarbons and Isopar® M contains about 84 wt % branched hydrocarbons. The Isopar range contains also between 6 and 15 wt % of cyclic alkanes, typically C6 with varying degrees of branching, and less than 1-2 wt % of aromatic contaminants. Isooctane (CAS No. 540-84-1); isododecane (CAS No. 141-70-8); isoeicosane (CAS No. 52845-07-5) and Isohexadecane (CAS No. 4390-04-9) are also considered as isoparaffinic hydrocarbon oils.

In a further embodiment, the perfume composition is substantially free of iso-paraffin, linear aliphatic hydrocarbons and saturated cyclic hydrocarbons, meaning it has no more than about 10 wt %, alternatively no more than about 5 wt %, alternatively no more than about 1 wt %, by weight of the composition.

Fragrance Ingredients

In accordance with one embodiment, fragrance ingredients for use in perfume compositions according to the present disclosure may be selected from natural products such as essential oils, absolutes, resinoids, resins, concretes, and synthetic perfume components such as hydrocarbons, alcohols, aldehydes, ketones, ethers, acids, acetals, ketals and nitriles, including saturated and unsaturated compounds, aliphatic, carbocyclic and heterocyclic compounds, or precursors of any of the above. Other examples of odorant compositions which may be used are described in H 1468 (United States Statutory Invention Registration, or in S. Arctander “Perfume and Flavor Chemicals: Volume 1, Allured Publishing Corporation 1969, or any later editions thereof, as well as the IFRA (International Fragrance Research Association) database, and RIFM (Research Institute of Fragrance Materials) database, each of which and hereby incorporated by reference in their entirety.

Suitable fragrance ingredients that are useful in membrane-based air freshener perfume compositions include, but are not limited to, 2-METHYL 2-PENTENOIC ACID (2-methyl-pent-2-enoic acid); ACETOIN (3-hydroxybutan-2-one); ACETOPHENONE EXTRA (acetophenone); AGRUMEX (2-(tert-butyl)cyclohexyl acetate); ALCOHOL C 9 NONYLIC (nonan-1-ol); ALDEHYDE C 10 DECYLIC (decanal); ALDEHYDE C 11 UNDECYLENIC (undec-10-enal); ALDEHYDE C 12 LAURIC (dodecanal); ALDEHYDE C 6 HEXYLIC (Hexan-1-ol); ALDEHYDE C 8 OCTYLIC (octanal); ALDEHYDE C 9 NONYLIC (nonanal); ALLYL AMYL GLYCOLATE (allyl 2-(isopentyloxy)acetate); ALLYL CYCLOHEXYL PROPIONATE (allyl 3-cyclohexylpropionate); AMBRETTOLIDE ((Z)-oxacycloheptadec-10-en-2-one); AMBROFIX® (3a,6,6,9a-tetramethyldodecahydronaphtho[2,1-b]furan); ANETHOLE SYNTHETIC ((E)-1-methoxy-4-(prop-1-en-1-yl)benzene); ANJERUK® (1-phenylethanethiol); APHERMATE (1-(3,3-dimethylcyclohexyl)ethyl formate); AUBEPINE PARA CRESOL (4-methoxybenzaldehyde); BENZALDEHYDE (benzaldehyde); BENZYL ACETATE (benzyl acetate); BICYCLO NONALACTONE (octahydro-2H-chromen-2-one); BORNEOL CRYSTALS ((1S,2S,4S)-1,7,7-trimethylbicyclo[2.2.1]heptan-2-ol); BORNYL ACETATE LIQUID ((2S,4S)-1,7,7-trimethylbicyclo[2.2.1]heptan-2-yl acetate); BUTYL ACETATE (butyl acetate); BUTYL BUTYRO LACTATE (1-butoxy-1-oxopropan-2-yl butanoate); BUTYL CYCLOHEXYL ACETATE PARA (4-(tert-butyl)cyclohexyl acetate); CARVONE LAEVO (2-methyl-5-(prop-1-en-2-yl)cyclohex-2-enone); CASHMERAN® (1,1,2,3,3-pentamethyl-2,3,6,7-tetrahydro-1H-inden-4(5H)-one); CASSYRANE® (5-tert-butyl-2-methyl-5-propyl-2H-furan); CEDRYL METHYL ETHER ((1R,6S,8aS)-6-methoxy-1,4,4,6-tetramethyloctahydro-1H-5,8a-methanoazulene); CETALOX® (3a,6,6,9a-tetramethyl-2,4,5,5a,7,8,9,9b-octahydro-1H-benzo[e][1]benzofuran); CINNAMALVA (cinnamonitrile); CINNAMIC ALDEHYDE (cinnamaldehyde); CINNAMYL ACETATE (cinnamyl acetate); CITRAL TECH ((E)-3,7-dimethylocta-2,6-dienal); CITRONELLOL (3,7-dimethyloct-6-en-1-ol); CITRONELLYL ACETATE (3,7-dimethyloct-6-en-1-yl acetate); CITRONELLYL OXYACETALDEHYDE (2-((3,7-dimethyloct-6-en-1-yl)oxy)acetaldehyde); CONIFERAN® (2-(tert-pentyl)cyclohexyl acetate); COUMARIN (2H-chromen-2-one); CYCLAL C (2,4-dimethylcyclohex-3-enecarbaldehyde); CYCLAMEN ALDEHYDE (3-(4-isopropylphenyl)-2-methylpropanal); CYCLOGALBANATE (allyl 2-(cyclohexyloxy)acetate); CYCLOHEXAL (4-(4-hydroxy-4-methylpentyl)cyclohex-3-enecarbaldehyde); CYMENE PARA (p-cymene); CYPRISATE (methyl 1,4-dimethylcyclohexanecarboxylate); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA ((E)-1-(2,6,6-trimethylcyclohex-3-en-1-yl)but-2-en-1-one); DECALACTONE GAMMA (5-hexyloxolan-2-one); DECENAL-4-TRANS ((E)-dec-4-enal); DIHYDRO EUGENOL (2-methoxy-4-propylphenol); DIHYDRO MYRCENOL (2,6-dimethyloct-7-en-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butanoate); DIMYRCETOL (2,6-dimethyloct-7-en-2-yl formate); DODECALACTONE DELTA (6-heptyltetrahydro-2H-pyran-2-one); EBANOL® ((E)-3-methyl-5-(2,2,3-trimethylcyclopent-3-en-1-yl)pent-4-en-2-ol); ELINTAAL (3-(1-ethoxyethoxy)-3,7-dimethylocta-1,6-diene); ETHYL BUTYRATE (ethyl butanoate); ETHYL CAPRONATE (ethyl hexanoate); ETHYL ISOAMYL KETONE (6-methylheptan-3-one); ETHYL ISOVALERATE (ethyl 3-methylbutanoate); ETHYL LINALOOL ((E)-3,7-dimethylnona-1,6-dien-3-ol); ETHYL MALTOL (2-ethyl-3-hydroxy-4H-pyran-4-one); ETHYL METHYL-2-BUTYRATE (ethyl 2-methylbutanoate); ETHYL OENANTHATE (ethyl heptanoate); ETHYL SAFRANATE (ethyl 2,6,6-trimethylcyclohexa-1,3-diene-1-carboxylate); ETHYL VANILLIN (3-ethoxy-4-hydroxybenzaldehyde); EUCALYPTOL ((1s,4s)-1,3,3-trimethyl-2-oxabicyclo[2.2.2]octane); EUGENOL (4-allyl-2-methoxyphenol); EVERNYL (methyl 2,4-dihydroxy-3,6-dimethylbenzoate); FENCHYL ALCOHOL ((1S,2R,4R)-1,3,3-trimethylbicyclo[2.2.1]heptan-2-ol); FLORALOZONE® (3-(4-ethylphenyl)-2,2-dimethylpropanal); FLORHYDRAL® (3-(3-isopropylphenyl)butanal); FLORIDILE® ((E)-undec-9-enenitrile); FLOROPAL® (2,4,6-trimethyl-4-phenyl-1,3-dioxane); FRESKOMENTHE® (2-(sec-butyl)cyclohexanone); FRUCTONE (ethyl 2-(2-methyl-1,3-dioxolan-2-yl)acetate); GALBANONE (1-(3,3-dimethylcyclohex-1-en-1-yl)pent-4-en-1-one); GARDENOL (1-phenylethyl acetate); GERANIOL ((E)-3,7-dimethylocta-2,6-dien-1-ol); HEDIONE (methyl 3-oxo-2-pentylcyclopentaneacetate); HELIOTROPINE CRYSTALS (benzo[d][1,3]dioxole-5-carbaldehyde); HEXENYL ACETATE CIS-3 (cis-hex-3-enyl acetate); HEXENYL-3-CIS BENZOATE ((Z)-hex-3-en-1-yl benzoate); HEXENYL-3-CIS SALICYLATE ((Z)-hex-3-en-1-yl 2-hydroxybenzoate); HEXYL ACETATE (hexyl acetate); HEXYL BUTYRATE (hexyl butanoate); HEXYL ISOBUTYRATE (hexyl isobutanoate); IONONE BETA ((E)-4-(2,6,6-trimethylcyclohex-1-en-1-yl)but-3-en-2-one); IRISONE PURE ((E)-4-(2,6,6-trimethylcyclohex-2-en-1-yl)but-3-en-2-one); ISO E SUPER® (1-(2,3,8,8-tetramethyl-1,2,3,4,5,6,7,8-octahydronaphthalen-2-yl)ethanone); ISOAMYL ACETATE (isopentyl acetate); ISOAMYL BUTYRATE (isopentyl butanoate); ISOCYCLOCITRAL (2,4,6-trimethylcyclohex-3-enecarbaldehyde); ISOEUGENOL ((E)-2-methoxy-4-(prop-1-en-1-yl)phenol); ISOMENTHONE DL (2-isopropyl-5-methylcyclohexanone); ISOPENTYL ISOVALERATE (isopentyl 3-methylbutanoate); ISOPROPYL-2 METHYL-4 THIAZOLE (2-isopropyl-4-methylthiazole); ISORALDEINE® 70 ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); JASMATONE® (2-hexylcyclopentanone); JAVANOL® ((1-methyl-2-((1,2,2-trimethylbicyclo[3.1.0]hexan-3-yl)methyl)cyclopropyl)methanol); KOHINOOL® (3,4,5,6,6-pentamethylheptan-2-ol); LABIENOXIME ((3E,6E)-2,4,4,7-tetramethylnona-6,8-dien-3-one oxime); LEMONILE® ((2E,6Z)-3,7-dimethylnona-2,6-dienenitrile); LIFFAROME® ((Z)-hex-3-en-1-yl methyl carbonate); LINALOOL (3,7-dimethylocta-1,6-dien-3-ol); LINALYL ACETATE (3,7-dimethylocta-1,6-dien-3-yl acetate); MACEAL (bicyclo[2.2.2]oct-5-ene-2-carboxaldehyde); MANZANATE (ethyl 2-methylpentanoate); MENTHOL (2-isopropyl-5-methylcyclohexanol); METHOXY PHENYL BUTANONE (4-(4-methoxyphenyl)butan-2-one); METHYL AMYL KETONE (heptan-2-one); METHYL ANTHRANILATE (methyl 2-aminobenzoate); METHYL CINNAMATE (methyl cinnamate); METHYL HEPTENONE (6-methylhept-5-en-2-one); METHYL HEXYL KETONE (octan-2-one); METHYL PAMPLEMOUSSE® (6,6-dimethoxy-2,5,5-trimethylhex-2-ene); MUSK C14 (1,4-dioxacyclohexadecane-5,16-dione); MYRCENE 90 (7-methyl-3-methyleneocta-1,6-diene); NEOFOLIONE ((E)-methyl non-2-enoate); NONANYL ACETATE (nonanyl acetate); ORANGER CRYSTALS (1-(2-naphtalenyl)-ethanone); OXANE® 50%/TEC (2-methyl-4-propyl-1,3-oxathiane); OXYOCTALINE FORMATE (2,4a,5,8a-tetramethyl-1,2,3,4,4a,7,8,8a-octahydronaphthalen-1-yl formate); PARADISAMIDE® (2-ethyl-N-methyl-N-(m-tolyl)butanamide); PEACH PURE (5-heptyldihydrofuran-2(3H)-one); PELARGOL (3,7-dimethyloctan-1-ol); PHENOXY ACETALDEHYDE 50 (2-phenoxyacetaldehyde); PHENYL ETHYL ALCOHOL (2-phenylethanol); PRUNOLIDE (5-pentyldihydrofuran-2(3H)-one); RADJANOL® ((E)-2-ethyl-4-(2,2,3-trimethylcyclopent-3-en-1-yl)but-2-en-1-ol); RASPBERRY KETONE (N112) (4-(4-hydroxyphenyl)butan-2-one); RESEDAL (2-(cyclohexylmethyl)-4,4,6-trimethyl-1,3-dioxane); RHUBAFURAN® (2,4-dimethyl-4-phenyltetrahydrofuran); STRAWBERRY PURE (ethyl methyl phenyl glycidate); SYLKOLIDE® ((E)-2-((3,5-dimethylhex-3-en-2-yl)oxy)-2-methylpropyl cyclopropanecarboxylate); TERPENYL ACETATE (2-(4-methylcyclohex-3-en-1-yl)propan-2-yl acetate); TERPINEOL PURE (2-(4-methylcyclohex-3-en-1-yl)propan-2-ol); TETRAHYDRO LINALOOL (3,7-dimethyloctan-3-ol); THYMOL CRYSTALS (2-isopropyl-5-methylphenol); TRANS-2-HEXENAL (E-hex-2-enal); TRICYCLAL (2,4-dimethylcyclohex-3-enecarbaldehyde); TRIFERNAL (3-phenylbutanal); TRIMOFIX O® (1-((2E,5Z,9Z)-2,7,8-trimethylcyclododeca-2,5,9-trien-1-yl)ethanone); UNDECAVERTOL ((E)-4-methyldec-3-en-5-ol); VANILLIN (4-hydroxy-3-methoxybenzaldehyde); ZINARINE® (2-(2,4-dimethylcyclohexyl)pyridine).

In a particular embodiment, the membrane-based air freshener perfume compositions according to the present disclosure include a fragrance ingredients, for example, AGRUMEX (2-(tert-butyl)cyclohexyl acetate); ALCOHOL C 9 NONYLIC (nonan-1-ol); ALDEHYDE C 10 (decanal); ALLYL AMYL GLYCOLATE (allyl 2-(isopentyloxy)acetate); AUBEPINE PARA CRESOL (4-methoxybenzaldehyde); BENZYL ACETATE (benzyl acetate); BORNYL ACETATE ((2S,4S)-1,7,7-trimethylbicyclo[2.2.1]heptan-2-yl acetate); CINNAMIC ALDEHYDE (cinnamaldehyde); CITRONELLOL (3,7-dimethyloct-6-en-1-ol); DELTA DAMASCONE ((E)-1-(2,6,6-trimethylcyclohex-3-en-1-yl)but-2-en-1-one); DIHYDRO MYRCENOL (2,6-dimethyloct-7-en-2-ol); DIMETHYL BENZYL CARBINYL BUTYRATE (2-methyl-1-phenylpropan-2-yl butanoate); DIMYRCETOL (2,6-dimethyloct-7-en-2-yl formate); ETHYL LINALOOL ((E)-3,7-dimethylnona-1,6-dien-3-ol); ETHYL 2-METHYL BUTYRATE (ethyl 2-methylbutanoate); ETHYL MALTOL (2-ethyl-3-hydroxy-4H-pyran-4-one); ETHYL OENANTHATE (ethyl heptanoate); ETHYL VANILLIN (3-ethoxy-4-hydroxybenzaldehyde); EUCALYPTOL ((1s,4s)-1,3,3-trimethyl-2-oxabicyclo[2.2.2]octane); EUGENOL (4-allyl-2-methoxyphenol); FENCHYL ALCOHOL ((1S,2R,4R)-1,3,3-trimethylbicyclo[2.2.1]heptan-2-ol); FLORALOZONE® (3-(4-ethylphenyl)-2,2-dimethylpropanal); FRESKOMENTHE® (2-(sec-butyl)cyclohexanone); FRUCTONE (ethyl 2-(2-methyl-1,3-dioxolan-2-yl)acetate); DECALACTONE gamma (5-hexyloxolan-2-one); GARDENOL (1-phenylethyl acetate); GERANIOL ((E)-3,7-dimethylocta-2,6-dien-1-ol); LINALOOL (3,7-dimethylocta-1,6-dien-3-ol), HEXENYL-3-CIS ACETATE ((Z)-hex-3-en-1-yl acetate); HEXYL ACETATE (hexyl acetate); HEXYL ACETATE (hexyl acetate); IONONE BETA ((E)-4-(2,6,6-trimethylcyclohex-1-en-1-yl)but-3-en-2-one); ISOAMYL ACETATE (isopentyl acetate); ISOAMYL BUTYRATE (isopentyl butanoate); ISORALDEINE 70 ((E)-3-methyl-4-(2,6,6-trimethylcyclohex-2-en-1-yl)but-3-en-2-one); JASMACYCLENE ((3aR,6S,7aS)-3a,4,5,6,7,7a-hexahydro-1H-4,7-methanoinden-6-yl acetate); LEMONILE® ((2E,6Z)-3,7-dimethylnona-2,6-dienenitrile); LINALYL ACETATE (3,7-dimethylocta-1,6-dien-3-yl acetate); MACEAL (bicyclo[2.2.2]oct-5-ene-2-carboxaldehyde); MANZANATE (ethyl 2-methylpentanoate); METHYL ANTHRANILATE (methyl 2-aminobenzoate); METHYL CINNAMATE (methyl cinnamate); METHYL HEPTENONE (6-methylhept-5-en-2-one); METHYL HEXYL KETONE (octan-2-one); METHYL PAMPLEMOUSSE (6,6-dimethoxy-2,5,5-trimethylhex-2-ene); NONANYL ACETATE (nonanyl acetate); PEACH PURE (5-heptyldihydrofuran-2(3H)-one); PELARGOL (3,7-dimethyloctan-1-ol); PRUNOLIDE (5-pentyldihydrofuran-2(3H)-one); RASPBERRY KETONE (4-(4-hydroxyphenyl)butan-2-one); TERPENYL ACETATE (2-(4-methylcyclohex-3-en-1-yl)propan-2-yl acetate); TERPINEOL PURE (2-(4-methylcyclohex-3-en-1-yl)propan-2-ol); TETRAHYDRO LINALOOL (3,7-dimethyloctan-3-ol); TRICYCLAL (2,4-dimethylcyclohex-3-enecarbaldehyde).

In a further embodiment, the membrane-based air freshener perfume compositions according to the present disclosure include more than 15 wt %, in another embodiment more than 20 wt % and in yet another embodiment more than about 25 wt % of alkyl alcohols having a chain length larger than about 5 carbon atoms, and terpene alcohols and alcohols derived from modified terpenes or terpene analogues, such as CITRONELLOL (3,7-dimethyloct-6-en-1-ol); DIHYDRO MYRCENOL (2,6-dimethyloct-7-en-2-ol); ETHYL LINALOOL ((E)-3,7-dimethylnona-1,6-dien-3-ol); FENCHYL ALCOHOL ((1S,2R,4R)-1,3,3-trimethylbicyclo[2.2.1]heptan-2-ol); GERANIOL ((E)-3,7-dimethylocta-2,6-dien-1-ol); LINALOOL (3,7-dimethylocta-1,6-dien-3-ol); ETHYL LINALOOL ((E)-3,7-dimethylnona-1,6-dien-3-ol); PELARGOL (3,7-dimethyloctan-1-ol); TETRAHYDRO LINALOOL (3,7-dimethyloctan-3-ol); TERPINEOL PURE (2-(4-methylcyclohex-3-en-1-yl)propan-2-ol); and the like; and ALCOHOL C 9 NONYLIC (nonan-1-ol); and the like.

Without wishing to be bound by theory, it is believed that membrane-based air fresheners having a membrane comprising an apolar non-porous polymer such as polyethylene, polypropylene or any polyolefin or polymer showing selective permeability such that the permeability to polar fragrance ingredients is lower that the permeability to apolar fragrance ingredients results in an increase in the transfer of fragrance ingredients through the membrane.

### Optional Ingredients

Membrane-based air freshener perfume compositions according to the present disclosure can further include texture modifying agents, such viscosifying agents or gelling agents, such as ETHOCEL. In a one embodiment, a membrane-based air freshener perfume composition may comprise from about 1 to about 3 wt %, in another embodiment from about 1.5 to about 2.5 wt % and in yet another embodiment from about 1.8 to about 2.0 wt % of a texture modifying agent.

The membrane-based air freshener perfume compositions according to the present disclosure can further include chelating agents, such as diethylene triamine penta (methylene phosphonic acid) derivatives, ethylene diamine tetraacetic acid and salts, polyamine carboxylic acid, and the like; antioxidants, such as pentaerythrityl tetra-(di-t-butyl hydroxyhydrocinnamate), 2,6-di-tert-butyl-4-methylphenol, and the like; UV filters, such as 2-Ethylhexyl (2E)-3-(4-methoxyphenyl)-2-propenoate, ethylhexyl methoxycinnamate, and the like; thickeners, such as hydrophobic silica and cellulose derivatives, for example ethyl cellulose, and the like; and dyes.

### EXAMPLES

The following examples are given solely for the purpose of illustration and are not to be construed as limitations of the present invention, as many variations of the invention are possible without departing from the spirit and scope of the present disclosure. Example 4 is a suitable perfume composition for membrane air fresheners according to the present disclosure. Examples 1-3 are comparative.

### Examples 1-4

### Perfume Compositions

A series of perfume compositions were prepared by mixing fragrance ingredients. The perfumes have the compositions reported in Table 1.

TABLE 1
Perfume compositions (Examples 1-4) for use in non-
porous polyethylene membrane-based air fresheners
(all numbers are in weight percent (wt %).

| | Exam- | Exam- | Exam- | Exam- |
|---|---|---|---|---|
| | ple 1 | ple 2 | ple 3 | ple 4 |
| LIQUID CARRIERS | | | | |
| ALKANES | 0.0 | 10.0 | 0.0 | 0.0 |
| ALKYL & TERPENE ESTERS | 77.8 | 7.7 | 31.3 | 0.0 |
| AROMATIC ESTERS | 0.0 | 37.7 | 13.8 | 0.0 |
| DOWANOL DPM¹ | 0.0 | 33.3 | 0.0 | 0.0 |
| DOWANOL DPGDME² | 0.0 | 0.0 | 0.0 | 40.0 |
| FRAGRANCE INGREDIENTS | | | | |
| ALKYL & TERPENE | 0.9 | 5.0 | 13.0 | 31.8 |
| ALCOHOLS | | | | |
| TERPENES | 0.0 | 0.0 | 5.4 | 10.0 |
| ALDEHYDES | 2.6 | 2.7 | 5.1 | 0.0 |
| AROMATIC ALCOHOLS | 0.3 | 0.0 | 1.4 | 5.0 |
| CYCLOALKYL ESTERS | 2.2 | 0.7 | 3.3 | 3.0 |
| OTHER ESTERS | 9.2 | 7.7 | 0.0 | 12.0 |
| ETHERS | 0.9 | 0.0 | 0.0 | 0.0 |
| KETALS | 0.1 | 0.0 | 0.5 | 0.0 |
| KETONES | 2.7 | 0.3 | 8.4 | 0.0 |
| LACTONES | 0.0 | 1.0 | 5.4 | 1.0 |
| MUSKS | 0.4 | 0.0 | 0.0 | 0.2 |
| NITRILES | 0.0 | 1.7 | 0.0 | 0.0 |
| OILS | 0.4 | 0.0 | 4.8 | 1.0 |
| OXIDES | 0.0 | 0.0 | 2.7 | 1.0 |
| VISCOSIFYING AGENT | 1.8 | 0.0 | 1.9 | 0.0 |

¹Dipropylene Glycol Mono Methyl Ether (supplied by Dow Chemicals)
²Dipropylene Glycol Dimethyl Ether (supplied by Dow Chemicals under the trademark PROGLYDE DMM)

In Table 1, the liquid carriers used in the Examples include: ISONONANYL ACETATE in Example 1; BENZYL ACETATE, ISOPAR L and DOWANOL DPM in Example 2; BENZYL ACETATE, LINALYL ACETATE and TERPENYL ACETATE in Example 3; and DIPROPYLENE GLYCOL DIMETHYL ETHER in Example 4. As apparent from Table 1, Example 4 has the lowest level of liquid carrier and the highest level of alkyl and terpene alcohol, while still having a high level of alkane and terpene (LIMONENE in this example).

### Examples 5-10

### Perfume Compositions

In order to demonstrate the benefits of the present disclosure, a series of compositions were prepared, using LINALOOL as terpene alcohol. The compositions are reported in Table 2.

TABLE 2
Perfume compositions (Examples 5-10) for use in membrane-based air fresheners (all numbers
are in weight percent (wt %).

| | | | | | | Example | VAPOR PRESSURE |
|---|---|---|---|---|---|---|---|
| | Example 5 | Example 6 | Example 7 | Example 8 | Example 9 | 10 | (mmHg @20° C.)⁶ |
| Fragrance | | | | | | | |
| Ingredients | | | | | | | |
| DIPENTENE¹ | 0% | 0% | 10% | 10% | 0% | 0% | 1.6 |
| LINALOOL² | 40% | 40% | 40% | 40% | 40% | 100% | 0.02 |
| Liquid Carriers | | | | | | | |
| DPGDME³ | 50% | 0% | 50% | 0% | 60% | 0% | 0.6 |
| DPM⁴ | 0% | 50% | 0% | 50% | 0% | 0% | 0.3 |
| ISOPAR L⁵ | 10% | 10% | 0% | 0% | 0% | 0% | 0.2 |

¹Fragrance ingredient (supplied by Givaudan as LIMONENE)
²Fragrance ingredient (supplied by Givaudan)
³Dipropylene Glycol Dimethyl Ether (supplied by Dow Chemicals under the trademark PROGLYDE DMM)
⁴Dipropylene Glycol Mono Methyl Ether (supplied by Dow Chemicals under DOWANOL brand)
⁵C11-C13 Isoparaffins (supplied by Exxon)
⁶Estimated values (ex http://www.thegoodscentscompany.com/, Dow Chemicals and Exxon)

The above compositions were placed in a passive, non-porous polyethylene membrane-based air freshener device, having an evaporation surface of 20 cm², operating at room temperature (about 25±2° C.) and the weight loss of the device was monitored as a function of time. Weight loss rates higher than 15% per 10 days were considered as acceptable (“Good”) for the sake of the application, while lower weight loss rates were considered as unacceptable (“Poor”). The weight loss rates are reported in Table 3.

TABLE 3
Weight loss rates (in weight percent)

| | Example 5 | Example 6 | Example 7 | Example 8 | Example 9 | Example 10 |
|---|---|---|---|---|---|---|
| 10 days | 30% | 12% | 30% | 10% | 20% | 9% |
| 20 days | 52% | 21% | 45% | 15% | 36% | 17% |
| | GOOD | POOR | GOOD | POOR | GOOD | POOR |

These results demonstrate the benefit of using DPGDME in passive membrane air freshener applications. As apparent from Example 7, DPGDME alone is an excellent carrier solvent for both DIPENTENE (aprotic ingredient) and LINALOOL (protic ingredient).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A perfume composition for use in non-porous membrane-based air fresheners comprising:
one or more fragrance ingredients; and
from about 5% to about 90% by weight of a liquid carrier having individual Hansen solubility parameters of: a dispersion solubility parameter (δ_{d}) less than about 17 MPa^{1/2}, a polar solubility parameter (δₚ) ranging from about 1 to about 7 MPa^{1/2}, and a hydrogen bond solubility parameter (δₕ) ranging from about 2 to about 7 MPa^{1/2}; a C log P lower than about 3.5; and a vapor pressure ranging from about 0.2 to about 1 mmHg.

2. The perfume composition according to claim 1, wherein the liquid carrier has individual Hansen solubility parameters of: a dispersion solubility parameter (δ_{d}) less than about 17 MPa^{1/2}, a polar solubility parameter (δₚ) ranging from about 1.8 to about 4 MPa^{1/2}, and a hydrogen bond solubility parameter (δₕ) ranging from about 3 to about 5 MPa^{1/2}; a C log P lower than about 1.0; and a vapor pressure ranging from about 0.4 to about 0.6 mmHg.

3. The perfume composition according to claim 1, wherein the composition is substantially free of hydrocarbons selected from the group consisting of iso-paraffins, linear aliphatic hydrocarbons and saturated cyclic hydrocarbons.

4. The perfume composition according to claim 1, wherein the composition is substantially free of protic glycol ethers.

5. The perfume composition according to claim 1, wherein the liquid carrier is a fully alkylated alkylene glycol according to formula (I) 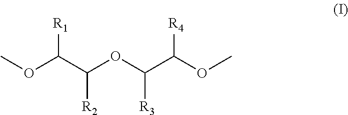
wherein R₁ is CH3 or H;
R₂ is H if R₁ is CH₃ or R₂ is CH₃ if R₁ is H;
R₃ is CH₃ or H; and
R₄ is H if R₃ is CH₃ or R₄ is CH₃ if R₃ is H.

6. The perfume composition according to claim 5, wherein the fully alkylated alkylene glycol is dipropylene glycol dimethyl ether (DPGDME).

7. The perfume composition according to claim 1, wherein the one or more fragrance ingredients include at least about 15%, by weight of the perfume composition of ingredients selected from the group consisting of alkyl alcohols having a chain length larger than about 5 carbon atoms, terpene alcohols, and alcohols derived from modified terpenes or terpene analogues.

8. The perfume composition according to claim 7, wherein the one or more fragrance ingredients are selected from the group consisting of 3,7-dimethyloct-6-en-1-ol; 2,6-dimethyloct-7-en-2-ol; (E)-3,7-dimethylnona-1,6-dien-3-ol; (1S,2R,4R)-1,3,3-trimethylbicyclo[2.2.1]heptan-2-ol; (E)-3,7-dimethylocta-2,6-dien-1-ol; 3,7-dimethylocta-1,6-dien-3-ol; (E)-3,7-dimethylnona-1,6-dien-3-ol; 3,7-dimethyloctan-1-ol; 3,7-dimethyloctan-3-ol; 2-(4-methylcyclohex-3-en-1-yl)propan-2-ol; nonan-1-ol; and mixtures thereof.

9. The perfume composition according to claim 7, wherein the one or more fragrance ingredients are selected from the group consisting of (6,6-dimethoxy-2,5,5-trimethylhex-2-ene); (ethyl 2-(2-methyl-1,3-dioxolan-2-yl)acetate); (3-(1-ethoxyethoxy)-3,7-dimethylocta-1,6-diene); ((Z)-hex-3-en-1-yl methyl carbonate); and mixtures thereof.

10. An air freshener device comprising:
(a) a housing; and
(b) a reservoir including a perfume composition and a permeable membrane through which a vapor of the perfume composition can permeate;
wherein the perfume composition includes one or more fragrance ingredients; and from about 5% to about 90% by weight of a liquid carrier having individual Hansen solubility parameters of: a dispersion solubility parameter (δ_{d}) less than about 17 MPa^{1/2}, a polar solubility parameter (δₚ) ranging from about 1 to about 7 MPa^{1/2}, and a hydrogen bond solubility parameter (δₕ) ranging from about 2 to about 7 MPa^{1/2}; a C log P lower than about 3.5; and a vapor pressure ranging from about 0.2 to about 1 mmHg.

11. The air freshener device according to claim 10, wherein the permeable membrane includes a gas permeable membrane comprising a polymer selected from the group consisting of polyolefins.

12. The air freshener device according to claim 10, wherein the perfume composition is substantially free of hydrocarbons selected from the group consisting of iso-paraffins, linear aliphatic hydrocarbons and saturated cyclic hydrocarbons.

13. The air freshener device according to claim 10, wherein the perfume composition is substantially free of protic glycol ethers.

14. The air freshener device according to claim 10, wherein the liquid carrier is a fully alkylated alkylene glycol according to formula (I) 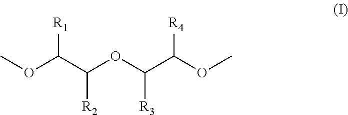
wherein R₁ is CH3 or H;
R₂ is H if R₁ is CH₃ or R₂ is CH₃ if R₁ is H;
R₃ is CH₃ or H; and
R₄ is H if R₃ is CH₃ or R₄ is CH₃ if R₃ is H.

15. The air freshener device according to claim 14, wherein the fully alkylated alkylene glycol is dipropylene glycol dimethyl ether (DPGDME).

16. The air freshener device according to claim 10, wherein the one or more fragrance ingredients include at least about 15%, by weight of the perfume composition of ingredients selected from the group consisting of alkyl alcohols having a chain length larger than about 5 carbon atoms, terpene alcohols, and alcohols derived from modified terpenes or terpene analogues.

17. The air freshener device according to claim 16, wherein the one or more fragrance ingredients are selected from the group consisting of 3,7-dimethyloct-6-en-1-ol; 2,6-dimethyloct-7-en-2-ol; (E)-3,7-dimethylnona-1,6-dien-3-ol; (1S,2R,4R)-1,3,3-trimethylbicyclo[2.2.1]heptan-2-ol; (E)-3,7-dimethylocta-2,6-dien-1-ol; 3,7-dimethylocta-1,6-dien-3-ol; (E)-3,7-dimethylnona-1,6-dien-3-ol; 3,7-dimethyloctan-1-ol; 3,7-dimethyloctan-3-ol; 2-(4-methylcyclohex-3-en-1-yl)propan-2-ol; nonan-1-ol; and mixtures thereof.

18. The air freshener device according to claim 10, wherein the perfume composition has an evaporation rate of at least 15% after 10 days.

19. A perfume composition for use in membrane-based air fresheners comprising:
at least about 25% by weight of one or more fragrance ingredients; and
from about 10% to about 60% by weight of a liquid carrier;
wherein the one or more fragrance ingredients is selected from the group consisting of alkyl alcohols having a chain length larger than about 5 carbon atoms, terpene alcohols, and alcohols derived from modified terpenes or terpene analogues; and wherein the liquid carrier is dipropylene glycol dimethyl ether (DPGDME).

20. The perfume composition according to claim 19, wherein the one or more fragrance ingredients are selected from the group consisting of 3,7-dimethyloct-6-en-1-ol; 2,6-dimethyloct-7-en-2-ol; (E)-3,7-dimethylnona-1,6-dien-3-ol; (1S,2R,4R)-1,3,3-trimethylbicyclo[2.2.1]heptan-2-ol; (E)-3,7-dimethylocta-2,6-dien-1-ol; 3,7-dimethylocta-1,6-dien-3-ol; (E)-3,7-dimethylnona-1,6-dien-3-ol; 3,7-dimethyloctan-1-ol; 3,7-dimethyloctan-3-ol; 2-(4-methylcyclohex-3-en-1-yl)propan-2-ol; nonan-1-ol; and mixtures thereof.
